(19) United States
(12) Patent Application Publication
(10) Pub. No.: US 2026/0256344 A1
(43) Pub. Date: Sep. 3, 2026

(54) Endoscopic Device with a Dynamic, User-Directed, Articulating Tip

(71) Applicant: THE REGENTS OF THE UNIVERSITY OF CALIFORNIA, Oakland, CA (US)

(72) Inventors: Dean Ehrlich, Los Angeles, CA (US); Jacob Rosen, Santa Cruz, CA (US); Alex Yihui Xu, Milpitas, CA (US)

(21) Appl. No.: 18/867,868

(22) PCT Filed: May 25, 2023
(86) PCT No.: PCT/US2023/067473

§ 371 Date: Nov. 21, 2024

Related U.S. Application Data
(60) Provisional application No. 63/345,943, filed on May 26, 2022.

(51) Int. Cl. A61B 1/005; A61B 1/00; A61B 1/018

(52) U.S. Cl. CPC A61B 1/0057; A61B 1/00042; A61B 1/00128; A61B 1/00137; A61B 1/0016; A61B 1/018

(57) ABSTRACT

The present invention provides an endoscopic device configure to allow a 360 degree rotation of the tip. In some embodiments, the endoscopic device of the present invention is configured to fit within a working channel of any known endoscopic device and allow independent movement of the tip of the known device, and the device of the present invention, for more precise targeting of a desired anatomic area.

### CROSS-REFERENCE TO RELATED APPLICATIONS

This application claims priority to U.S. Provisional Patent Application No. 63/345,943, filed May 26, 2022, the contents of which are incorporated by reference herein in its entirety.

### BACKGROUND OF THE INVENTION

Endoscopic evaluation of the GI tract is the primary procedure of the practicing gastroenterologist. There are many ways to perform endoscopy. The two main types of endoscopy are esophagogastroduodenoscopy (EGD) and colonoscopy. However, there are many other endoscopic procedure types, including but not limited to, Single-Balloon Enteroscopy, Double-Balloon Enteroscopy, Endoscopic Retrograde Cholangiopancreatography, and Endoscopic Ultrasound. Each of these procedures requires a different type of endoscope. The endoscope contains a “working channel,” sometimes also called “Instrument Channel” or “Biopsy Port.” Through this channel, the practitioner can feed a device through the endoscope to perform diagnostic and therapeutic procedures within the GI tract. The inserted device is operated in real-time by the practitioner or an accompanying GI technician from outside of the body, while simultaneously viewing the movement of the device on the video screen. Numerous devices already exist for use in the GI tract, including biopsy forceps, metal clips, cautery devices, metal snares, needles, among others. Each device is typically attached on the distal end (within the body) to a long, thin, catheter or wire that traverses through the endoscope, and ends with a handle on the proximal end (that stays outside the body).

One of the challenges for the GI practitioner is the ability to properly position the inserted device in the exact desired location and orientation within the lumen of the GI tract. The ability to properly position the device is paramount to the success of the procedure. At present, the primary method of positioning the device is by manipulation of the endoscope tip. The device typically rests just beyond the distal tip of the endoscope. The distal tip of the endoscope (within the patient) can be curved approximately 180 degrees using controls on the endoscope control head. This movement of the endoscope tip, in turn, moves the device. As the tip of the endoscope moves, the device moves as well.

However, there are many times during endoscopy when the desired area to place the device is not feasibly attained because of the limits of the rotational capability of the endoscope tip in relation to specific patient anatomy. For example, the desired area may not be able to be viewed head-on which limits the ability to properly use a device (e.g., removing a polyp in the right colon or placing a clip on an ulcer in the posterior duodenal bulb). In other instances, there may be extreme difficulty maintaining stable endoscopic position during colonoscopy which lessens precision of the procedure (e.g., snare polypectomy at the hepatic flexure). There are other occasions when properly positioning the device to the desired area of intervention is possible, but takes significant time to achieve and becomes ergonomically unfavorable for the practitioner (e.g., realigning a polyp on the side wall to the 6 o'clock position, in order to perform a snare polypectomy). At times, due to anatomic limitations, the practitioner must even use the device “blind,” meaning that the only way to reach a desired area of interest with the device is when the area of interest is momentarily out of view from the camera. These are just a few examples of situations in which the limits of requiring the device to be positioned solely by manipulation of the endoscope tip are apparent.

Thus, there is a need in the art for an endoscopic device to allow independent control of the movement of the endoscope tip and the tip of the device that feeds through the working channel of the endoscope. The present invention meets this need.

### SUMMARY OF THE INVENTION

Aspects of the present invention relate to a device having an elongated tubular body having a proximal end opening, a distal end opening, an outer surface and an inner surface forming a lumen therethrough from the proximal to distal end openings, a plurality of rings each having an inner diameter and an outer diameter, wherein the outer surface of the body is affixed to the inner diameter of each ring, and wherein each ring has at least one hole passing therethrough between the inner diameter and the outer diameter, a cap positioned over top of the distal end of the body, one or more actuators, and at least one linear wire having a first end and a second end, wherein the first end is connected to the cap, the second end is connected to the one or more actuators and wherein the at least one linear wire passes through the at least one hole in each of the plurality of rings.

In some embodiments, the body has a flexible portion configured to be bent softly and elastically by movement of the at least one linear wire. In some embodiments, the distance between each two adjacent rings along the body ranges between 0.5 mm to 250 cm. In some embodiments, the device includes three parallel linear wires. In some embodiments, the three parallel linear wires are spaced 120 degrees apart around the lumen. In some embodiments, the device further includes a splitter positioned at the proximal end of the body. In some embodiments, the cap has an end opening in fluid communication with the lumen of the body. In some embodiments, the end opening of the cap has a sharp interior portion.

In some embodiments, the one or more actuators includes a base comprising a bottom surface having a plurality of holes and at least one side wall extending longitudinally from the base, wherein the plurality of holes are configured to receive the plurality of parallel linear wires, a holder rotatably connected to the at least one side wall and having an opening in the middle, and a joystick having a bottom plate having a front end and a back end, wherein the bottom plate is positioned within the opening of the holder and is rotatably connected to the holder at the front end and the back end and wherein the bottom plate is connected to the plurality of parallel linear wires, and wherein the holder and the joystick can rotate around two perpendicular axis and are configured to allow a 360 degree rotation of the distal end of the lumen.

In some embodiments, the device further comprises a locking mechanism configured to lock the position of the holder or the joystick in place. In some embodiments, the locking mechanism is selected from the group consisting of a ratchet and pawl mechanism, a roller clutch mechanism, a hydraulic mechanism, and an electric mechanism. In some embodiments, the locking mechanism includes a plate having a teethed portion and a pawl, wherein the plate is positioned between the bottom plate and the holder, and the pawl is positioned on the at least one side wall and is configured to engage the teethed portion to lock the actuator in place.

In some embodiments, the at least one side wall is connected to the holder through a swivel stud. In some embodiments, the base has a second opening. In some embodiments, the one or more actuators further includes a spring positioned within the second opening, and wherein the spring is connected to a plate at a first end and to a pin at a second end. In some embodiments, the holder has two side walls.

In some embodiments, the at least one side wall has a second opening having a spring positioned thereinto, wherein the spring is connected to a plate at a first end towards the holder and to a pin at the second end, such that pushing the pin causes protrusion of the plate towards the holder to stop the holder from any further movement. In some embodiments, the one or more actuators includes one or more motors. In some embodiments, the device further includes a sleeve positioned around the outer surface of the lumen from the proximal end to the distal end.

Aspects of the present invention relate to a system, having a flexible, elongated body having a proximal end opening, a distal end opening and lumen connected to the proximal and distal end openings, a cap connected to a distal end of the body, and one or more actuators connected to the cap by a plurality of wires, wherein the one or more actuators is configured to control angular or directional movement of the distal end of the body by pulling or pushing at least one of the plurality of wires, and wherein the lumen is sized for at least one medical device to be inserted through the proximal end opening and through the lumen.

In some embodiments, the system further includes a plurality of flanges positioned along the length of the body, such that the plurality of wires passes through each of the flanges. In some embodiments, the one or more actuators includes one or more motors. In some embodiments, the system further includes a bracket positioned along the length of the body, with one or more flanges extending outward from the bracket, each flange comprising flat sidewalls extending out to a distal recess configured to fixedly and rotatably attach one or more pulley wheels, each pulley wheel configured to guide a wire from the plurality of wires.

In some embodiments, the one or more motors fixedly attach to one or more flat sidewalls on the flanges. In some embodiments, the one or more motors further include a stabilizing bracket extending out from the motors, configured to make contact with the flexible elongated body and prevent lateral and rotational movement of the motors with respect to the body. In some embodiments, the stabilizing bracket further has a recess configured to fixedly and rotatably attach a spindle, each spindle for connection to and rotatable winding of a wire from the plurality of wires.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 is a perspective side view of an exemplary endoscopic device of the present invention.

FIG. 2 is an exploded view of an exemplary endoscopic device of the present invention.

FIG. 3 depicts a perspective top view of an exemplary endoscopic device of the present invention.

FIG. 4 depicts an exemplary splitter of the present invention.

FIG. 5 depicts an exemplary handle attached to the first opening on the splitter of the present invention.

FIG. 6 depicts a perspective view of an exemplary actuator of the present invention.

FIG. 7 depicts a perspective view of an exemplary actuator of the present invention.

FIG. 8 depicts a side view of another exemplary actuator of the present invention.

FIG. 9 depicts a perspective view of an exemplary actuator of the present invention.

FIG. 10 depicts a top view of an exemplary actuator of the present invention.

FIG. 11 depicts a perspective view of an exemplary actuator of the present invention, wherein the holder is rotating around the a-axis.

FIG. 12 comprising FIGS. 12A and 12B, depict exploded views of exemplary actuators of the present invention.

FIG. 13 depicts a side view of another exemplary actuator of the present invention.

FIG. 14 depicts a top view of an exemplary actuator of the present invention.

FIG. 15 depicts a top view of an exemplary actuator of the present invention.

FIG. 16 depicts a ratchet and pawl locking mechanism of an exemplary actuator of the present invention.

FIG. 17 depicts an exemplary system of the present invention.

FIG. 18 depicts an exemplary system of the present invention.

FIG. 19 depicts an exemplary system of the present invention.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of endoscopy. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. “About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6. This applies regardless of the breadth of the range.

The present invention provides an endoscopic device configured to allow a 360 degree rotation of its tip. In some embodiments, the endoscopic device allows more precise targeting of a desired anatomic area, in situations where a straight device is limited. In some embodiments, the endoscopic device can reduce the time required to position the endoscope. In some embodiments, the endoscopic device can allow accurate visualization of anatomic areas that may be in the blind-spot of the endoscopist. In some embodiments, the endoscopic device can reduce ergonomically unfavorable positioning by the practitioner. In some embodiments, the endoscopic device of the present invention may be used as an add-on device compatible with any other known endoscopic device. In some embodiments, the endoscopic device of the present invention may be used in any field including but not limited to bronchoscopy (pulmonary diseases), laparoscopy (surgery), ureteroscopy (gynecology), cystoscopy (urology), cardiac catheterization, interventional radiology, vascular surgery, etc. In some embodiments, the endoscopic device can be used for removing a polyp in the right colon or placing a clip on an ulcer in the posterior duodenal bulb. In some embodiments, the endoscopic device can be used for realigning a polyp on the side wall to the 6 o'clock position, in order to perform a snare polypectomy. In some embodiments, the endoscopic device of the present invention may be used in plumbing, drilling and/or HVAC applications.

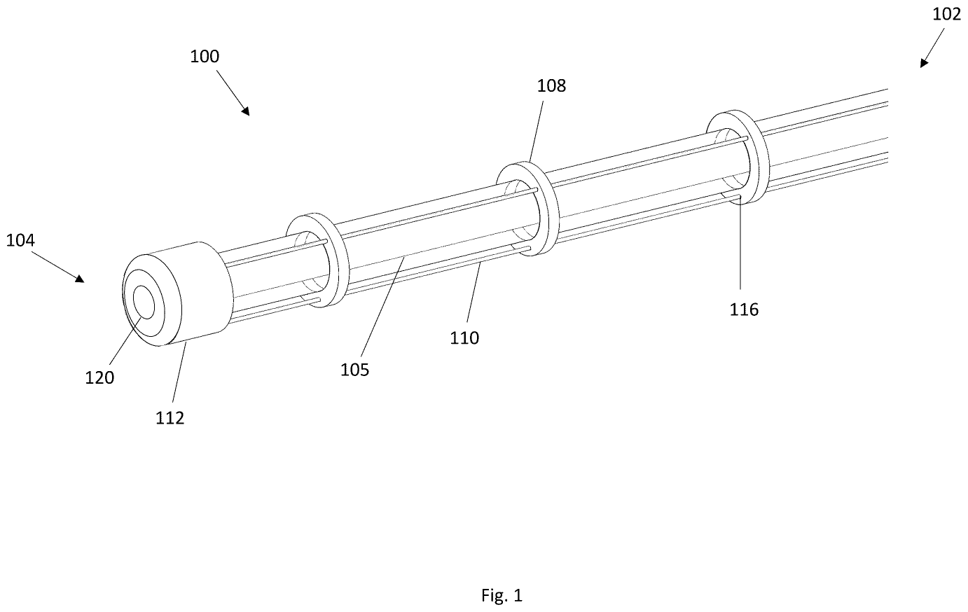

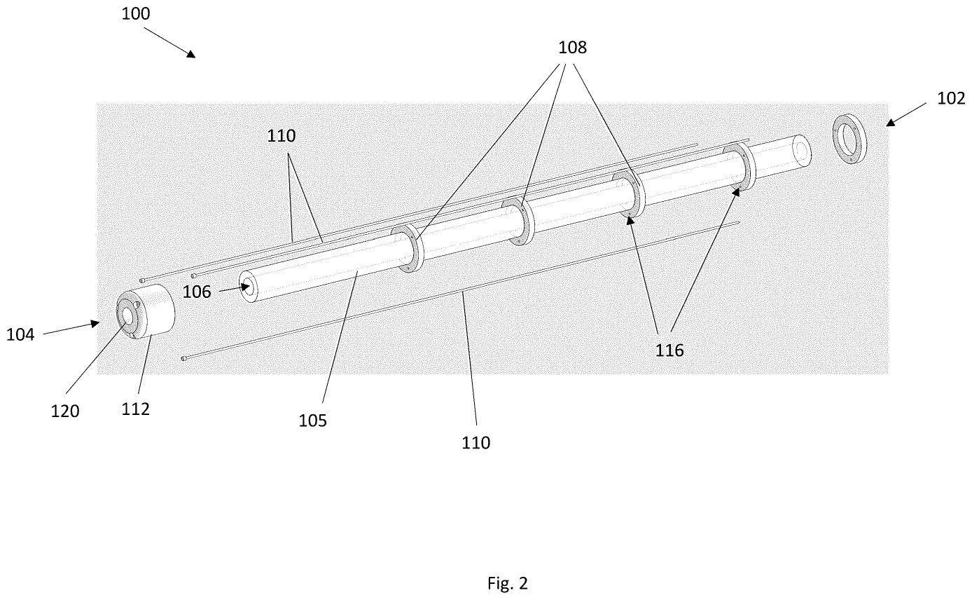

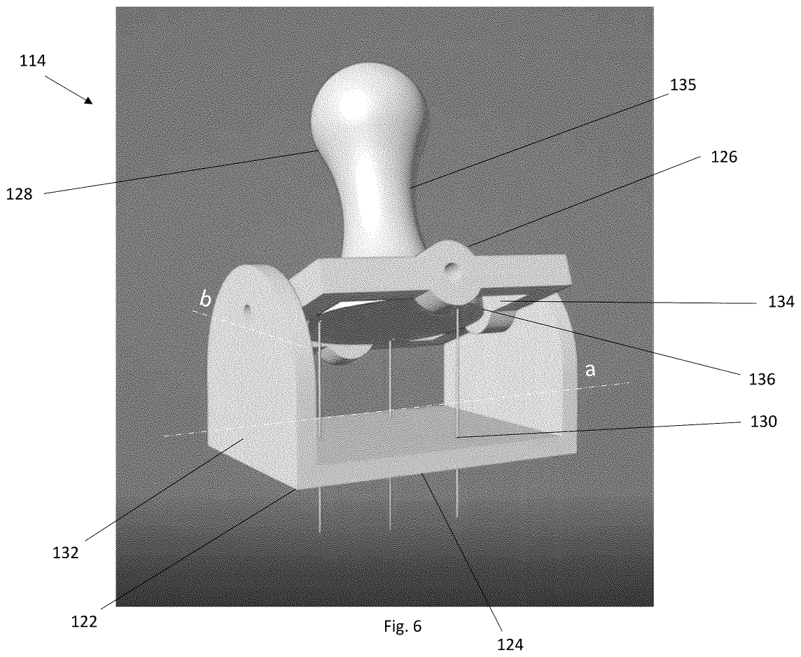

Referring now to FIGS. 1-3, an exemplary endoscopic device 100 of the present invention is shown. Device 100 comprises a proximal end 102 and a distal end 104 of an elongated tubular-shaped body 105 forming a lumen 106 therethrough, a plurality of flanges or rings 108, at least one linear wire 110, a distal cap 112 and one or more actuators 114 (see FIG. 6).

Lumen 106 is formed within body 105 along its length and is accessible from end openings at proximal and distal ends 102 and 104. Lumen 106 is configured to allow insertion and removal of any surgical instrument known to one skilled in the art including but not limited to a snare, forceps, hemoclip, bipolar cautery, injection needle, knife, a wire, sphincterotome, retrieval net, radiofrequency ablation, argon plasma coagulation, hemostasis sprays, cryotherapy, stent deployment, endoscopic ultrasound, artificial intelligence probes to detect polyps, etc. In some embodiments, lumen 106 may have any suitable diameter and length known to one skilled in the art based on various considerations, including the desired bodily passage within which the medical device is intended to be used. In some embodiments, lumen 106 may have a length ranging between 0.5 mm- 250 cm. In some embodiments, lumen 106 may have a diameter ranging between 0.5-10 mm. In some embodiments, the surrounding material of body 105 forming lumen 106 may be made with any suitable material known to one skilled in the art based on various considerations, including but not limited to the desired flexibility of body 105. Example materials considered suitable to form body 105 may include, but are not limited to, biocompatible materials, materials that can be made biocompatible, metals such as stainless steel, titanium, nickel-titanium alloys (e.g., Nitinol), polymers, polyvinyl chloride (PVC), rubber, nylon, polyethylene, polyurethane, polytetrafluoroethylene (PTFE), ePTFE, silicone, coiled materials, braided materials, and any other material considered suitable for a particular application. In some embodiments, body 105 may have a flexible portion which can be bent relatively softly and elastically by external force. In some embodiments, body 105 may have variable stiffness throughout its length. In one exemplary embodiment, body 105 may have a more flexible portion closer to proximal end 102 to allow more tortuosity in movement of the tip.

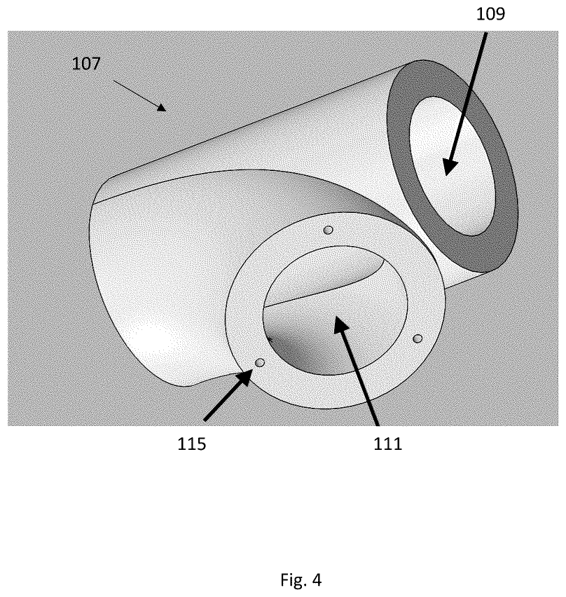

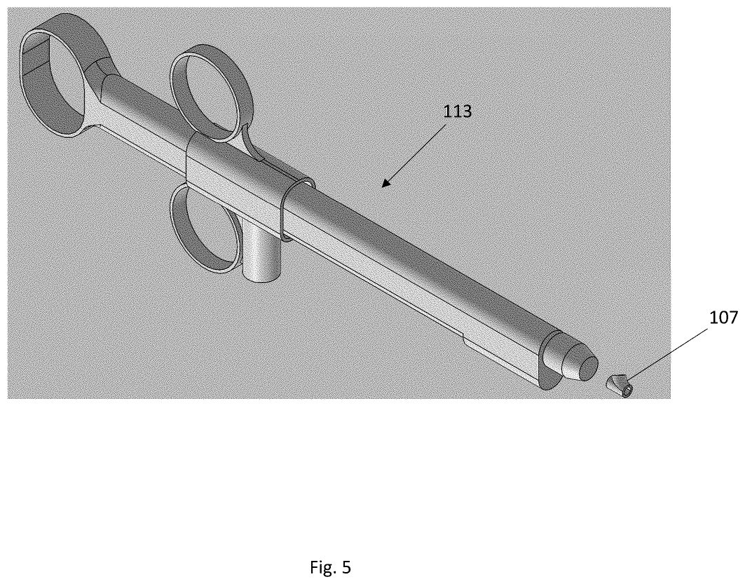

In some embodiments, body 105 forming lumen 106 may be connected to a splitter 107 at or near proximal end 102 (FIG. 4). Splitter 107 comprises a first opening 109 and a second opening 111. First opening 109 is fluidly connected to lumen 106 and configured to allow insertion and removal of any surgical instrument known to one skilled in the art, including but not limited to a snare. In some embodiments, first opening 109 may be connected to a handle 113, which may be the handle of any insertable surgical instrument understood by those in the art at or near proximal end 102 (FIG. 5). Second opening 111 is connected to actuator 114. As shown in FIG. 4, opening 111 is formed from a material having a thickness defined by an outer diameter, an inner diameter, and at least one hole 115 positioned between the outer diameter and the inner diameter. At least one hole 115 is configured to receive at least one linear wire 110 therethrough. In some embodiments, at least one hole 115 may have any diameter known to one skilled in the art. The number of holes 115 me be different than or commensurate with the number of linear wires 110 used. In various embodiments, second opening 111 may have one, two or three holes 115. In some embodiments, second opening 111 may have more than three holes 115. In some embodiments, first opening 109 and second opening 111 have the same diameter. In some embodiments, first opening 109 has a larger diameter than second opening 111. In some embodiments, first opening 109 has a smaller diameter than second opening 111. In some embodiments, body 105 with lumen 106 may be directly connected to actuator 114 at or near proximal end 102 without the need to use a splitter.

Plurality of flanges or rings 108 are positioned on the outer surface of body 105. Plurality of flanges or rings 108 have an inner diameter sized to fit around body 105 and an outer diameter ranging between 0.5-10 mm. Flanges or rings 108 may be affixed to the outer surface of body 105. In some embodiments, plurality of rings 108 are positioned parallel to each other along the length of body 105. In some embodiments, the distance between each two adjacent or subsequent rings 108 may be variable or may be equal along any portion of the length of body 105. In other embodiments, the distance between any two adjacent or subsequent flanges or rings may be ranging between 0.5 mm-250 cm. In some embodiments, plurality of rings 108 may be made with any suitable material known to one skilled in the art including but not limited to metals such as stainless steel, titanium, nickel-titanium alloys (e.g., Nitinol), polymers, polyvinyl chloride (PVC), rubber, nylon, polyethylene, polyurethane, polytetrafluoroethylene (PTFE), ePTFE, silicone, coiled materials, braided materials, etc. In other embodiments, the flanges or rings 108 may be molded or otherwise a single unit with body 105. In still other embodiments, flanges or rings may be slidably adjustable in position along the length of body 105. In still other embodiments, one or more flanges 108 may simply protrude from a portion of the outer surface of body 105, and do not wrap around the entire cross-sectional circumference of body 105.

Plurality of flanges or rings 108 comprise a plurality of holes 116 positioned within the protruding wall, for example between their inner diameter and the outer diameter, and are configured to receive at least one linear wire 110 therethrough. The number of holes 116 in each flange or ring 108 may be different than or commensurate with the number of linear wires 110. In some embodiments, plurality of rings 108 may comprise one, two or three holes 116 in each ring 108. In some embodiments, plurality of rings 108 may comprise more than three holes 116 in each ring 108.

The at least one linear wire 110 is connected at one end to cap 112 at distal end 104 and is further connected at the other end to actuator 114 at or near proximal end 102. In certain embodiments, device 100 may comprise one, two or three linear wires 110 spaced 120 degrees apart around the cross-sectional circumference of body 105. In some embodiments, device 100 may comprise more than three parallel linear wires 110. While there is no limitation to the number of wires 110 used, the benefit of fewer linear wires is seen in minimizing bulkiness and cost of the overall device, while the benefit of numerous linear wires may be improved specificity in articulation. When multiple wires 110 are used, any two adjacent wire may be of a variable distance about the cross-sectional circumference of body 105, or they may be equidistant from each other about the cross-sectional circumference of body 105.

In some embodiments, at least one linear wire 110 may have any suitable diameter and length known to one skilled in the art. In some embodiments, at least one linear wire 110 may have the same diameter throughout its length. In some embodiments, plurality of parallel linear wires 110 may have different diameters throughout its length. In some embodiments, at least one linear wire 110 may be made with any suitable material known to one skilled in the art including but not limited to metals such as stainless steel, titanium, nickel-titanium alloys (e.g., Nitinol), polymers, polyvinyl chloride (PVC), rubber, nylon, polyethylene, polyurethane, polytetrafluoroethylene (PTFE), ePTFE, silicone, coiled materials, braided materials, etc.

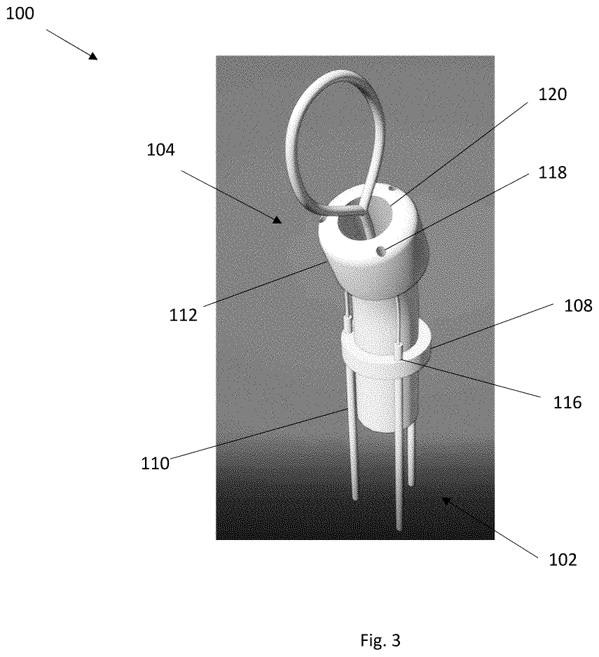

Cap 112 is positioned at distal end 104 of body 105. In some embodiments, cap 112 fits overtop and covers at least a portion of the outer surface of body 105 from distal end 104. Each of wires 110 is engaged or affixed to cap 112 by any mechanism known to those skilled in the art. For example, wires 110 may pass into and through at least one hole 118, configured to receive at least one linear wire 110 therethrough (FIG. 3). At least one hole 118 on cap 112 is aligned with at least one hole 116 positioned on plurality of rings 108. In certain embodiments, cap 112 may comprise an opening 120 that is in fluid communication with lumen 106. In some embodiments, opening 120 may have a sharp interior configured to more effectively cut biological tissues, such as polyps. In some embodiments, opening 120 has a diameter that is smaller than the diameter of lumen 106. In some embodiments, opening 120 has a diameter that is larger than the diameter of lumen 106. In some embodiments, opening 120 has a diameter equal to the diameter of lumen 106. In some embodiments, cap 112 may be made from any material known to one skilled in the art including but not limited to metals such as stainless steel, titanium, nickel-titanium alloys (e.g., Nitinol), polymers, polyvinyl chloride (PVC), rubber, nylon, polyethylene, polyurethane, polytetrafluoroethylene (PTFE), ePTFE, silicone, coiled materials, braided materials, etc. In some embodiments, body 105 has a flexible structure which can be bent relatively softly and elastically by external force applied to cap 112 from at least one linear wire 110.

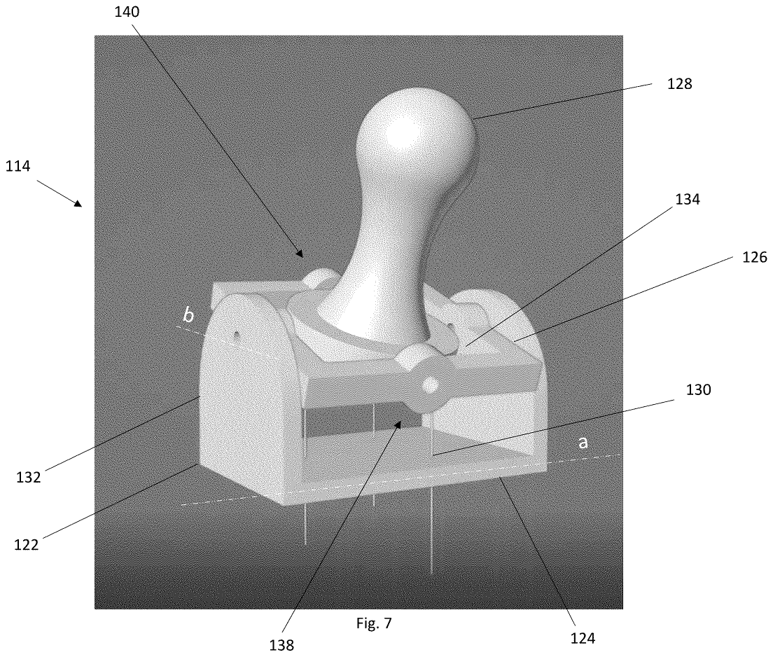

Referring now to FIG. 6 and FIG. 7, an exemplary actuator 114 of the present invention is shown. Actuator 114 comprises a base 122, a holder 126 and a joystick 128 and is configured to allow a tilting and rotating operation. Base 122 comprises a bottom plate 124 having at least one hole 130 and two side walls 132 extending longitudinally from bottom plate 124. At least one hole 130 is aligned with at least one hole 118 on cap 112, at least one hole 116 on plurality of rings 108, at least one hole 115 on second opening 111 and are configured to receive at least one linear wire 110 therethrough. Holder 126 comprises an opening 134 and is rotatably connected to two side wall 132 such that holder 126 is able to rotate around the a-axis. In some embodiments, holder 126 may be connected to two side walls 132 with any mechanism known to one skilled in the art. In some embodiments, holder 126 may have any shapes known to one skilled in the art including but not limited to rectangular, square, etc. In some embodiments, opening 134 may have any shapes known to one skilled in the art including but not limited to rectangular, square, etc.

Joystick 128 comprises an elongated shaft 135 and a bottom plate 136. Bottom plate 136 is positioned within opening 134 and is connected to at least one linear wire 110. Bottom plate 136 is rotatably connected to holder 126 at a front end 138 and a back end 140, such that joystick 128 can rotate around the b-axis. In some embodiments, bottom plate 136 may be connected to holder 126 with any mechanism known to one skilled in the art. In some embodiments, bottom plate 136 may have any shape known to one skilled in the art including but not limited to circular, square, rectangular, etc. In some embodiments, elongated shaft 135 may have any height known to one skilled in the art. In some embodiments, elongated shaft 135 may have any shape known to one skilled in the art including but not limited to cylindrical. In some embodiments, a-axis and b-axis are perpendicular to each other.

In some embodiments, one or more actuators 114 may comprise buttons or toggles to trigger a tilting and rotating operation. Although the one or more actuators 114 are described as various joysticks mechanically connected to the at least one wires 110, it should be appreciated that actuators 114 may comprise one or more motorized and/or rotating components to drive movement of wires 110. This may include servo motors, brushed DC motors, brushless DC motors, gearsets, pulleys, windings, bearings, rollers and the like.

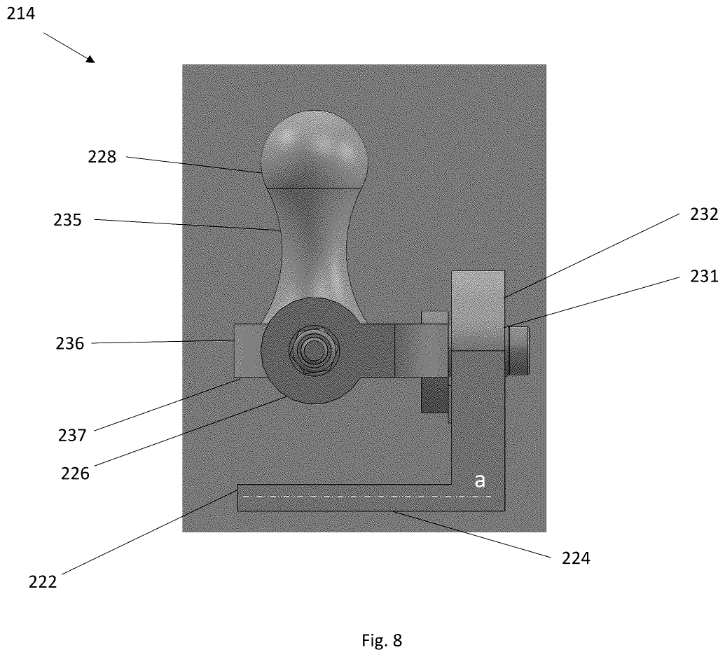

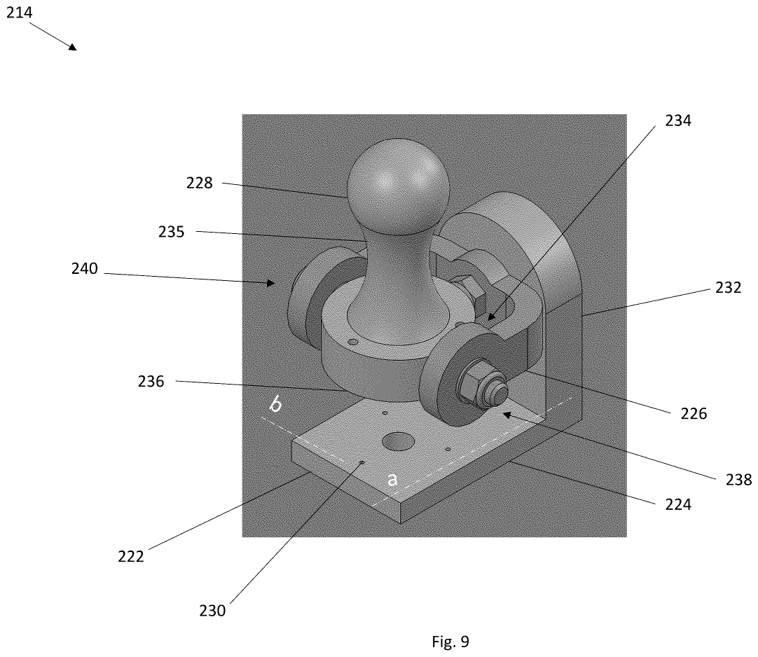

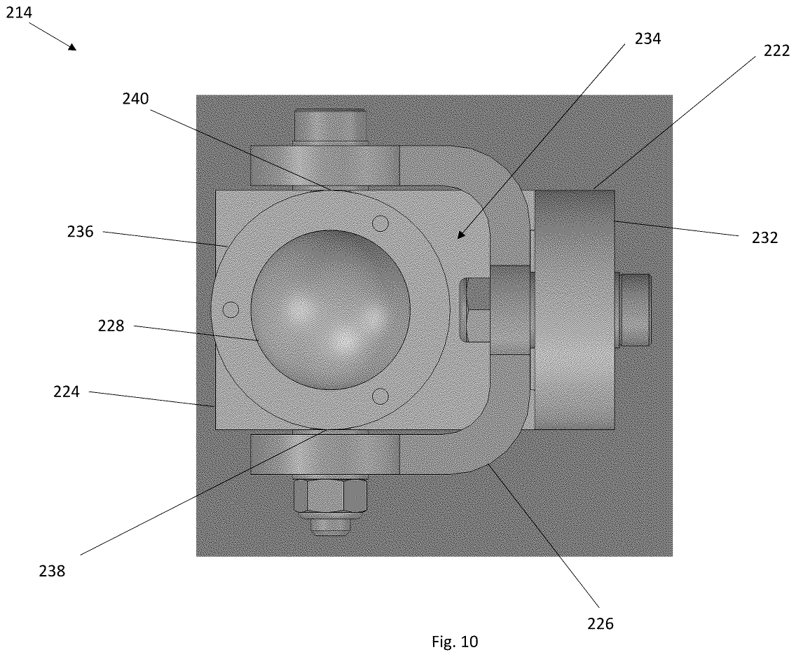

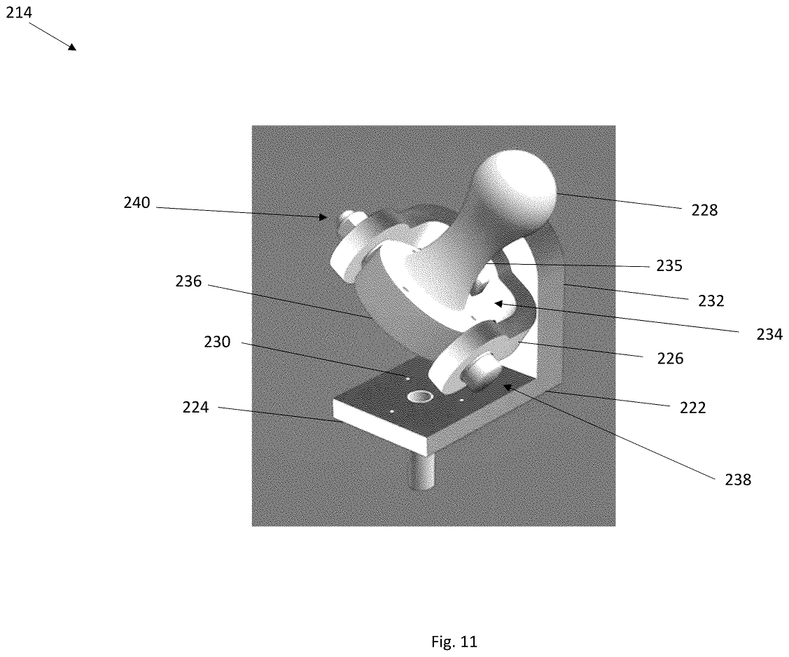

Referring now to FIG. 8 through FIG. 12, another exemplary actuator 214 of the present invention is shown. Actuator 214 comprises a base 222, a holder 226 and a joystick 228 and is configured to allow a tilting and rotating operation. Base 222 comprises a bottom plate 224 having at least one hole 230 and at least one side wall 232 extending longitudinally from bottom plate 224 and comprising an opening 231. At least one hole 230 is aligned with at least one hole 118 on cap 112, at least one hole 116 on plurality of rings 108, at least one hole 115 on second opening 111 of splitter 107 and is configured to receive at least one linear wire 110 therethrough.

Holder 226 comprises an opening 234 and is rotatably connected to at least one side wall 232 such that holder 226 is able to rotate around the a-axis. In some embodiments, holder 226 may be connected to at least one side wall 232 through any mechanism known to one skilled in the art that allows rotation of holder 226 around the a-axis. In some embodiments, holder 226 may be connected to at least one side wall 232 through insertion of a swivel stud through opening 231. In some embodiments, swivel stud may be secured in place through a bolt and a plurality of washers positioned around the stud. In some embodiments, holder 226 may have any shapes known to one skilled in the art including but not limited to rectangular, square, c-shaped, etc. In some embodiments, opening 234 may have any shapes known to one skilled in the art including but not limited to rectangular, square, circular, etc.

Joystick 228 comprises an elongated shaft 235 and a bottom plate 236. Bottom plate 236 comprises a bottom end 237, a first opening 238 and second opening 240, wherein first opening 238 and second opening 240 are fluidly connected to each other. Bottom plate 236 is positioned within opening 234 and rotatably connected to holder 226 by positioning a nut extending from first opening 238 to second opening 240, such that joystick 228 can rotate around the b-axis. In some embodiments, the nut may be secured in place by adding a bolt behind holder 226. In some embodiments, at least one washer is placed around the nut before placing it in first opening 238. In some embodiments, at least one washer is placed around the nut after the nut is passed through second opening 240 and before being secured with a bolt. In some embodiments, at least one washer may be positioned anywhere along the length of the nut.

Bottom end 237 is connected to at least one linear wire 110, such that any movement from joystick 228 translates into movement of at least one linear wire 110 and eventually distal end 104 of device 100. In some embodiments, bottom plate 236 may have any shape known to one skilled in the art including but not limited to circular, square, rectangular, etc. In some embodiments, elongated shaft 235 may have any height known to one skilled in the art. In some embodiments, elongate shaft 235 may have any shape known to one skilled in the art including but not limited to cylindrical.

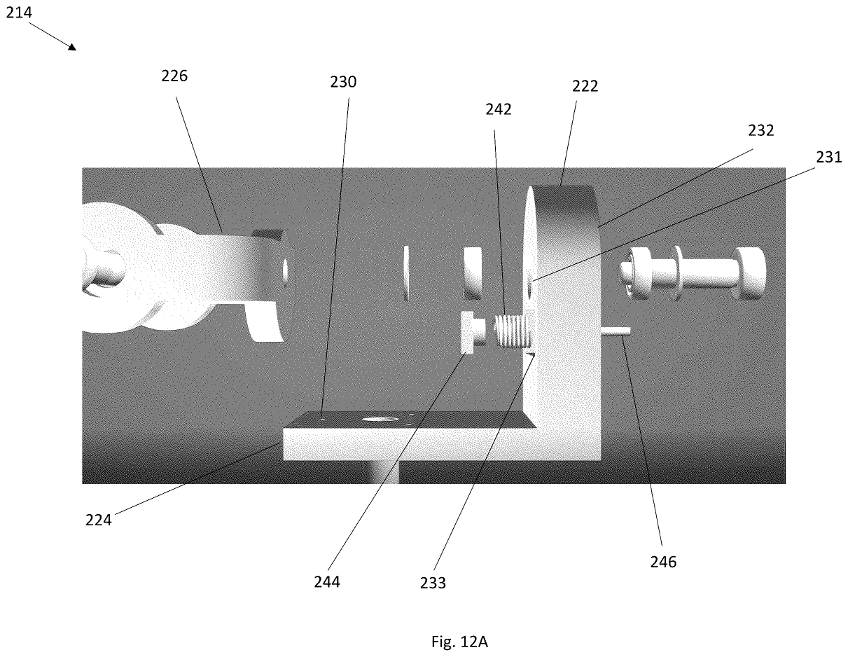

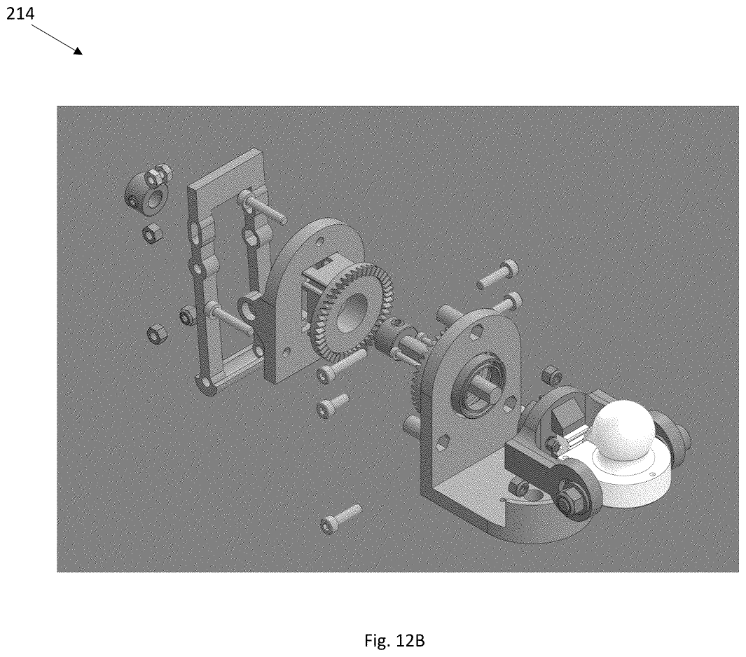

At least one side wall 232 may further comprise a second opening 233 (FIG. 12A). In some embodiments, second opening 233 may be positioned below opening 231. In some embodiments, second opening 233 may comprise a spring 242 positioned within the wall of base 222. Spring 242 is connected to a plate 244 at a first end and to a pin 246 at a second end, such that pushing pin 246 causes protrusion of plate 244 towards holder 226. This acts as a locking mechanism to stop any further movements of holder 226.

Movement of actuator 114 and 214 around both a-axis and b-axis transfers to movement of at least one linear wire 110 and leads to respective movement of distal end 104 of device 100.

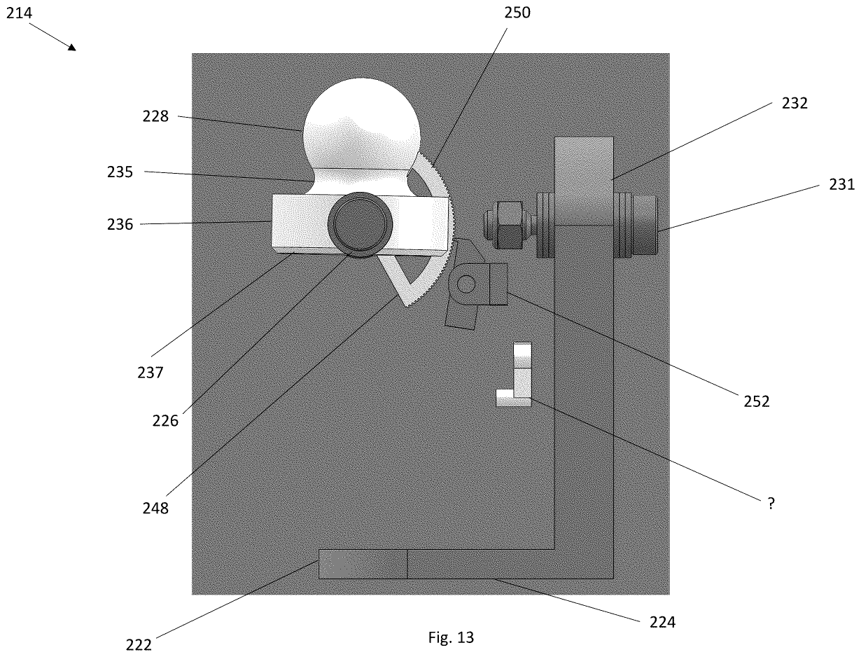

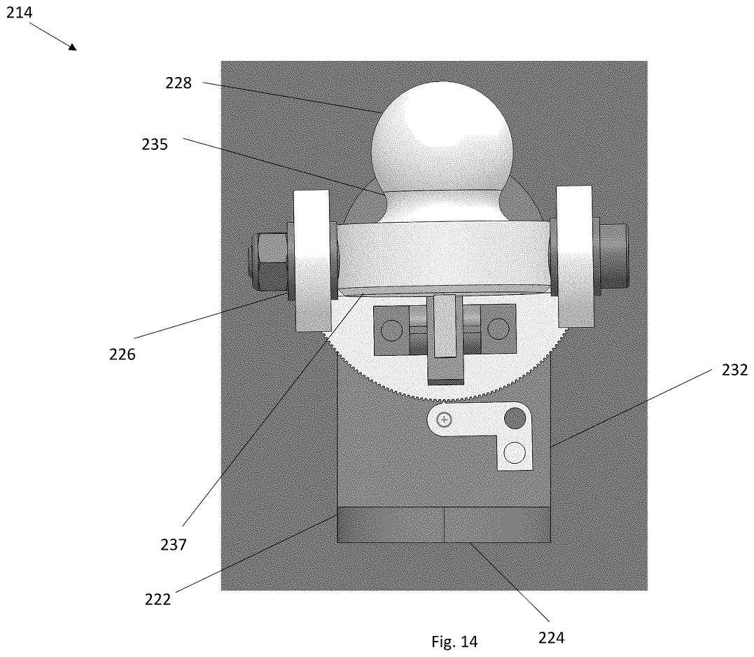

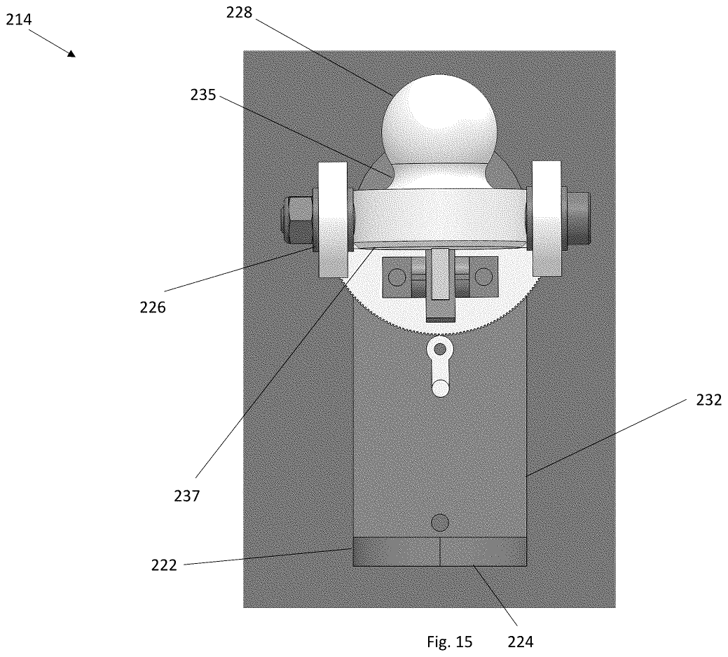

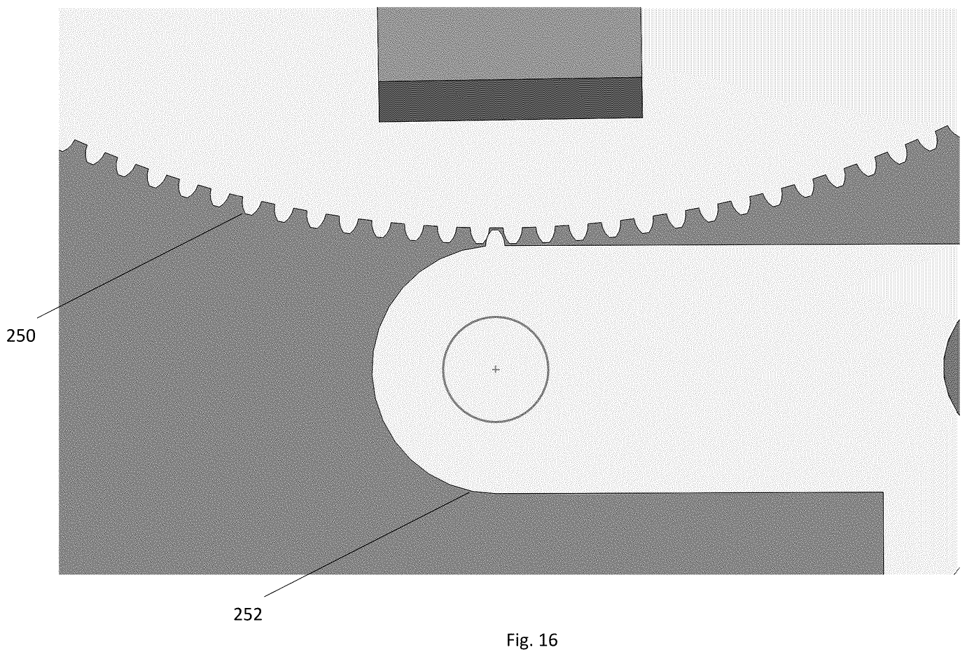

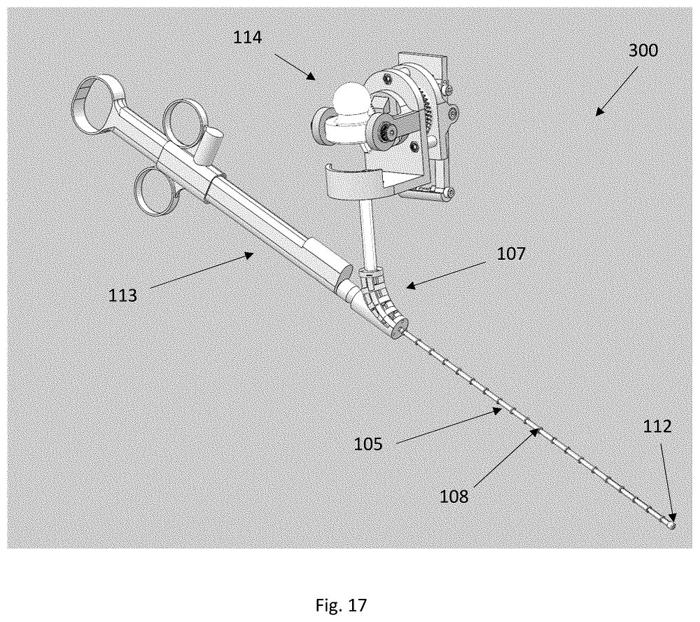

Referring now to FIG. 13 through FIG. 16, actuator 214 may further comprise a rachet locking mechanism for stopping or locking the holder, joystick or both. Rachet locking mechanism comprises a plate 248 having a teethed portion 250 and a pawl 252 (a and/or b). Plate 248 is positioned between bottom plate 236 and holder 226. Pawl 252 is positioned on at least one side wall 232 and is configured to engage teethed portion 250 to lock actuator 214 in a desired position.

Teethed portion 250 and pawl 252 of the ratchet mechanism may have any geometry that serves to perform the ratcheting function, i.e., the shapes of the teeth and pawl must be such that the teeth are able to slide or ratchet past the pawl in one direction, but not in the other direction without applying a force to disengage the teeth. In other words, the surfaces of pawl 252 and teethed portion 250 slide past one another in one direction but catch and lock when moved in the other direction. In some embodiments, teethed portion 250 and pawl 252 may have corresponding sawtooth serrations or profiles. The size and depth of pawl 252 and teethed portion 250 affect the step size of the graduated movement, and thus by controlling the length of the teeth along the longitudinal axis the step size of the graduated movement can be controlled.

In some embodiments, the locking mechanism may be any other mechanism including but not limited to a roller clutch mechanism, a hydraulic mechanism, an electric mechanism, etc.

In yet another embodiment, an endoscopic system 300 is shown. System 300 includes elongated tubular body 105 having a lumen therethrough, a plurality of flanges or rings 108 on the outer surface and along the length of body 105, a distal cap 112 at the distal end of body 105, a splitter 107 at the proximal end of body 105, an actuator 114 connected to a first branch from splitter 107, and an insertion device or handle 113 connected to a second branch from splitter 107. Wires (not shown but described elsewhere herein as wires 110) connect at one end to actuator 114 and pass through splitter 107, along the outer surface of body 105 through flanges or rings 108, and connect to cap 112 at the far end of the wires. In practice, the actuator pulls and/or pushes selected wires to manipulate cap 112, thereby steering cap 112 in any desired direction as body 105 is advanced to the target location in a patient, subject or other environment. Once body 105 has been steered into position, a medical device may be inserted from handle 113 through the lumen of body 105 as needed for a medical procedure.

In some embodiments, device 100 may be used as a standalone device. In some embodiments, device 100 may be inserted through a working channel of any endoscopic device known to one skilled in the art, and is configured to allow independent movement of an endoscope and device 100 from each other.

In some embodiments, device 100 may further comprise a sleeve (not shown) positioned around the external surface of the device from proximal end 102 to distal end 104. In some embodiments, the sleeve is configured to allow easier introduction and removal of the device. In some embodiments, the sleeve encloses and/or protects flanges 116 and wires 110. In some embodiments, the sleeve may be made with any suitable material known to one skilled in the art including but not limited to metals such as stainless steel, titanium, nickel-titanium alloys (e.g., Nitinol), polymers, polyvinyl chloride (PVC), rubber, nylon, polyethylene, polyurethane, polytetrafluoroethylene (PTFE), ePTFE, silicone, coiled materials, braided materials, etc.

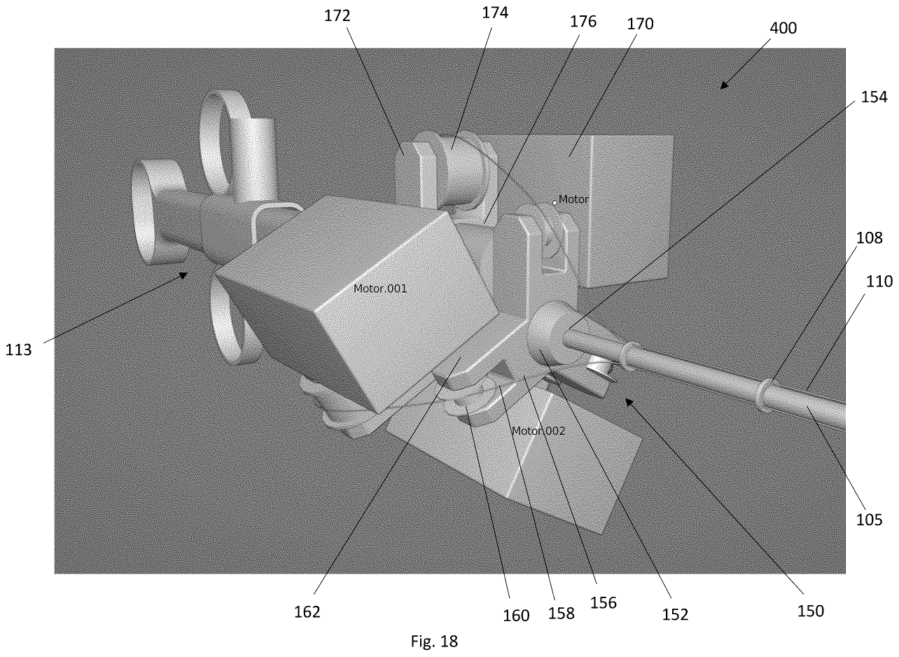

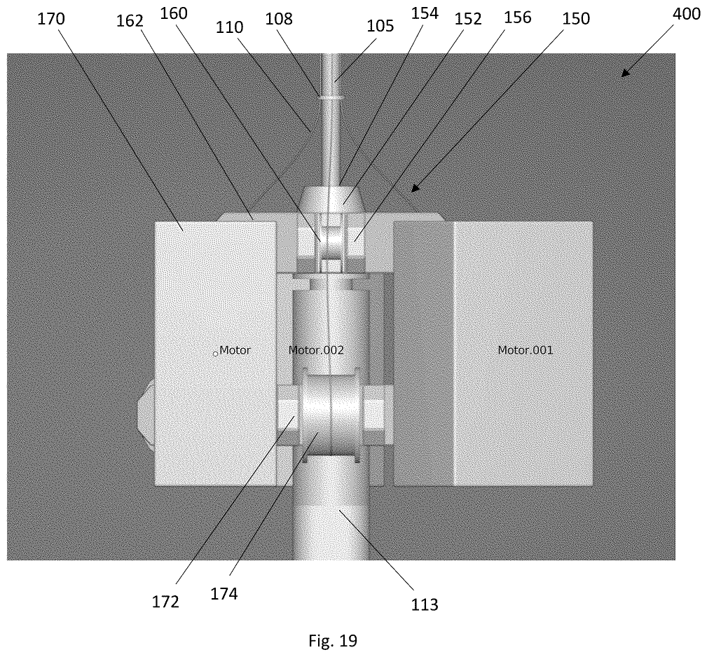

Referring now to FIGS. 18 and 19, an endoscopic system 400 is shown. System 400 includes elongated tubular body 105 having a lumen therethrough, a plurality of flanges or rings 108 on the outer surface and along the length of body 105, a distal cap at the distal end of body 105 (not shown but described elsewhere herein as distal cap 112), a bracket 150 positioned on the outer surface and along the length of body 105, one or more motors 170 connected to bracket 150, and an insertion device or handle 113 connected to elongated tubular body 105. Similar to other described embodiments of actuators 114, one or more motors 170 as configured are able to perform functionally the same directional movements of the existing embodiments.

In some embodiments, bracket 150 is positioned on the outer surface and along the length of body 105 and comprises a body region 152 with an opening 154 passing through the body region, the opening configured to receive at least a portion of body 105 through the opening. In some embodiments, one or more flanges 156 extend outward from body region 152 of bracket 150, and comprise a distal recess 158 configured to receive and rotatably attach one or more pulley wheels 160. In some embodiments, flanges 156 further comprise a sidewall region 162 configured to fixedly attach one or more motors 170.

In some embodiments, one or more motors 170 comprise one or more stabilizing brackets 172 extending outward laterally from the one or more motors 170, wherein the bracket is configured to receive and rotatably attach one or more spindles 174. Stabilizing brackets 172 further comprise a rounded base 176 configured to rest on and make contact with a portion of handle 113. In some embodiments, stabilizing brackets 172 and/or rounded base 176 prevent bracket 150 from lateral and/or rotational movement when attached to body 105 and/or handle 113.

In some embodiments, pulleys 160 and spindles 174 are aligned with at least one hole 116 on plurality of rings 108, and at least one hole on the distal cap (not shown but described elsewhere herein as hole 118 and distal cap 112) and configured to receive at least one linear wire 110 therethrough and thereon. Wires 110 connect at one end to spindles 174 and pass over pulleys 160, along the outer surface of body 105 through flanges or rings 108, and connect to the distal cap at the far end of the wires. Rotation of motors 170 transfers movement to at least one linear wire 110 and leads to respective movement of distal end 104 of device 100. In practice, the motors 170 and spindles 174 pull selected wires to manipulate cap 112 by rotating in a first direction, thereby steering cap 112 in any desired direction as body 105 is advanced to the target location in a patient, subject or other environment. In some embodiments, once tension on the respective wire 110 is released, from motors 170 rotating in a second direction, body 105 and/or steering cap 112 return to their original configuration and/or a straight position. In some embodiments, motors 170 rotating in the second direction push the respective wire 110 and cap 112 to any desired direction. Once body 105 has been steered into a position, a medical device may be inserted from handle 113 through the lumen of body 105 as needed for a medical procedure.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A device comprising:
an elongated tubular body having a proximal end opening, a distal end opening, an outer surface and an inner surface forming a lumen therethrough from the proximal to distal end openings;
a plurality of rings each having an inner diameter and an outer diameter, wherein the outer surface of the body is affixed to the inner diameter of each ring, and wherein each ring has at least one hole passing therethrough between the inner diameter and the outer diameter;
a cap positioned over top of the distal end of the body;
one or more actuators; and
at least one linear wire having a first end and a second end, wherein the first end is connected to the cap, the second end is connected to the one or more actuators and wherein the at least one linear wire passes through the at least one hole in each of the plurality of rings.

2. The device of claim 1, wherein the body comprises a flexible portion configured to be bent softly and elastically by movement of the at least one linear wire.

3. The device of claim 1, wherein the distance between each two adjacent rings along the body ranges between 0.5 mm to 250 cm.

4. The device of claim 1, wherein the device comprises three parallel linear wires.

5. The device of claim 4, wherein the three parallel linear wires are spaced 120 degrees apart around the lumen.

6. The device of claim 1, further comprising a splitter positioned at the proximal end of the body.

7. The device of claim 1, wherein the cap comprises an end opening in fluid communication with the lumen of the body.

8. The device of claim 7, wherein the end opening of the cap comprises a sharp interior portion.

9. The device of claim 1, wherein the one or more actuators comprises:
a base comprising a bottom surface having a plurality of holes and at least one side wall extending longitudinally from the base, wherein the plurality of holes are configured to receive the plurality of parallel linear wires;
a holder rotatably connected to the at least one side wall and comprising an opening in the middle; and
a joystick comprising a bottom plate having a front end and a back end, wherein the bottom plate is positioned within the opening of the holder and is rotatably connected to the holder at the front end and the back end and wherein the bottom plate is connected to the plurality of parallel linear wires; and
wherein the holder and the joystick can rotate around two perpendicular axis and are configured to allow a 360 degree rotation of the distal end of the lumen.

10. The device of claim 9, further comprising a locking mechanism configured to lock the position of the holder or the joystick in place.

11. The device of claim 10, wherein the locking mechanism is selected from the group consisting of: a ratchet and pawl mechanism, a roller clutch mechanism, a hydraulic mechanism, and an electric mechanism.

12. The device of claim 9, wherein the locking mechanism comprises a plate having a teethed portion and a pawl, wherein the plate is positioned between the bottom plate and the holder, and the pawl is positioned on the at least one side wall and is configured to engage the teethed portion to lock the actuator in place.

13. The device of claim 9, wherein the at least one side wall is connected to the holder through a swivel stud.

14. The device of claim 9, wherein the base comprises a second opening.

15. The device of claim 11, wherein the one or more actuators further comprises a spring positioned within the second opening, and wherein the spring is connected to a plate at a first end and to a pin at a second end.

16. The device of claim 9, wherein the holder comprises two side walls.

17. The device of claim 9, wherein the at least one side wall comprises a second opening having a spring positioned thereinto, wherein the spring is connected to a plate at a first end towards the holder and to a pin at the second end, such that pushing the pin causes protrusion of the plate towards the holder to stop the holder from any further movement.

18. The device of claim 1, wherein the one or more actuators comprises one or more motors.

19. The device of claim 1, further comprising a sleeve positioned around the outer surface of the lumen from the proximal end to the distal end.

20. A system, comprising:
a flexible, elongated body having a proximal end opening, a distal end opening and lumen connected to the proximal and distal end openings;
a cap connected to a distal end of the body; and
one or more actuators connected to the cap by a plurality of wires;
wherein the one or more actuators is configured to control angular or directional movement of the distal end of the body by pulling or pushing at least one of the plurality of wires; and
wherein the lumen is sized for at least one medical device to be inserted through the proximal end opening and through the lumen.

21. The system of claim 20, further comprising a plurality of flanges positioned along the length of the body, such that the plurality of wires passes through each of the flanges.

22. The device of claim 20, wherein the one or more actuators comprises one or more motors.

23. The system of claim 22, further comprising a bracket positioned along the length of the body, with one or more flanges extending outward from the bracket, each flange comprising flat sidewalls extending out to a distal recess configured to fixedly and rotatably attach one or more pulley wheels, each pulley wheel configured to guide a wire from the plurality of wires.

24. The system of claim 23, wherein the one or more motors fixedly attach to the flat sidewalls on the flanges.

25. The device of claim 22, wherein the one or more motors further comprise a stabilizing bracket extending out from the motors, configured to make contact with the flexible elongated body and prevent lateral and rotational movement of the motors with respect to the body.

26. The device of claim 25, wherein the stabilizing bracket further comprises a recess configured to fixedly and rotatably attach a spindle, each spindle for connecting to and rotatably winding a wire from the plurality of wires.
